(19) United States
(12) Patent Application Publication
(10) Pub. No.: US 2005/0118101 A1
(43) Pub. Date: Jun. 2, 2005

(54) Beta-homolysine conjugates and their use as transport enhancer

(76) Inventors: Olivier Loiseleur, Saint-Louis (FR); Daniel Kaufmann, Therwil (CH); Stephan Abel, Weil am Rhein (DE); Hans-Michael Burger, Allschwil (CH); Mark Meisenbach, Durmenach (FR); Beat Schmitz, Allschwil (CH); Gottfried Sedelmeier, Schallstadt (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/502,533

(22) PCT Filed: Jan. 27, 2003
(86) PCT No.: PCT/EP03/00803

(30) Foreign Application Priority Data
Jan. 28, 2002 (GB) 02018810
Feb. 7, 2002 (GB) 02028751

(51) Int. Cl. A61K 49/00; C07K 14/47; C07H 21/04; C12Q 1/68

(52) U.S. Cl. 424/9.6; 530/409; 536/25.32; 435/6

(57) ABSTRACT

The present invention relates to a conjugate that comprises a) at least one compound to be delivered into or across a biological carrier, b) a delivery-enhancing transporter comprising at least 4 β-homolysine residues, especially between 4 and 25 β-homolysine residues, c) optionally a linker between the components a) and b), and d) optionally a labelling unit; to the salts of such conjugates; to a pharmaceutical composition comprising said conjugate; to a method for delivery of a compound into or across a biological barrier, the method comprising contacting the barrier with said conjugate; and to a process for the preparation of certain conjugates.

The present invention provides conjugates and methods that enhance the delivery of drugs and other compounds into and across a biological barrier.

The practical application of biomolecules such as oligonucleotides, antibodies, functional peptides or proteins is generally hampered by lack of transport of such biomolecules into cells and their inefficiency in reaching their pharmacological target. Due to their size and hydrophilic nature, most biomolecules do not readily translocate across biological barriers, such as the lipid brayer of biological membranes. Surprisingly it was now found that β-homolysine polymers comprising at least 4, β-homolysine units are able to cross biological barriers and can deliver compounds conjugated to such polymers into or across a biological barrier.

Hence, the present invention relates to a conjugate (CONJUGATE) that comprises a) at least one compound (CARGO) to be delivered into or across a biological barrier; b) a delivery-enhancing transporter (SHUTTLE) comprising at least 4 β-homolysine residues; c) optionally a linker (LINKER) between the components a) and b); and d) optionally a labelling unit (A); and to the salts thereof.

Advantageously, β-homolysine polymers are not subject to enzymatic hydrolysis. Furthermore, contrary to α-homolysine polymers, β-homolysine polymers are known not to be toxic. These properties render β-homolysine polymers suitable for use as a SHUTTLE for the enhanced transport of pharmacologically active compounds into warm-blooded animals' membranes and cells. Hence, the CONJUGATES find use in therapeutic, prophylatic and diagnostic applications. The SHUTTLE can carry a diagnostic or biologically active agent into and across one or more layers of skin or other epithelial tissue or across endothelial tissues such as the blood brain barrier. Furthermore, such conjugates can be employed in in vitro assays and tests in order to enhance and visualize, e.g., the transport of macromolecules into cells and cell cultures.

In a preferred embodiment of the invention, the CONJUGATE has a structure selected from the group of structures (I) to (IV),
A-SHUTTLE-CARGO-(CO)—Y (II),
A-CARGO-SHUTTLE-(CO)—Y (II),
SHUTTLE-LINKER-CARGO (III), and
SHUTTLE-LINKER-CARGO-(CO)—Y (IV),
wherein Y is OR or NR₁R₂ and wherein R, R₁ and R₂ independently of each other represent hydrogen or alkyl. In such conjugates of formula (I) to (IV), A can represent a labelling unit selected from biotinyl, fluorescein-5-yl-NH—C(S)— and fluorescein-5-yl-NH—C(S)—NH—CH₂-Dᵣ-Eᵤ-Gₚ-CH₂—C(O)—, wherein D, E and G are independently of each other selected from CH₂, O or NH, under the proviso that not two heteroatoms are bonded to each other, and p, r and u are independently of each other an integer between 0 and 10.

The CONJUGATES, and especially those of structures (I) to (IV), can be prepared by methods known in the art. Suitable methods of preparation are described, e.g., by R. Eritja, “Synthesis of Oligonudeotide-Peptide Conjugates and Nucleopeptides”, in “Solid-Phase Synthesis”, Ed. S. A. Kates and F. Albericio, 2000, Marcel Dekker, Inc., New York, Basel, Ch. 12, pp. 529 to 548, and by P. Lloyd-Williams, F. Albericio and E. Giralt in “Chemical Approaches to the Synthesis of Peptides and Proteins, CRC Press, Boca Raton, 1997, in particular in Ch. 4.4, pp. 175 -207, and in the publications cited therein, respectively. In particular, carbamate, ester, thioether, disulfide and hydrazone linkages are generally easy to form and suitable for most applications.

The CARGO can be a biomolecule selected from the group consisting of oligonucleotides, e.g., antisense sequences for single- or double-stranded targets, peptides, proteins and antibodies. In one embodiment of the invention, the CARGO is a pharmacologically active compound or a diagnostic imaging or contrast agent. Such CARGO includes, but is not limited to, antihistamines, glucocorticoids, retinoids, cytotoxics, like aromatase inhibitors, antiestrogens, topoisomerase I inhibitors, topoisomerase II inhibitors, microtubule active agents, alkylating agents, antimetabolites, platin compounds, compounds decreasing the protein kinase activity, antiangiogenic compounds, gonadorelin agonists, antiandrogens, bisphosphonates and trastuzumab, and immunosuppressive drugs, like cyclosporins, tacrolimus or rapamycin.

The term “delivery-enhancing” as used herein relates to an increase in the amount and/or rate of delivery of a CARGO into and/or across a biological barrier.

In particular, the present invention provides conjugates represented by formula V 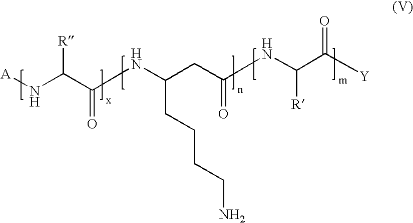
wherein
A represents an oligonucleotide, peptide, protein, a diagnostic imaging or contrast agent, H, biotinyl, fluorescein-5-yl-NH—C(S)— or fluorescein-5-yl-NH—C(S)—NH—CH₂-Dᵣ-Eᵤ-Gₚ-CH₂—C(O)—, wherein D, E and G are independently of each other selected from CH₂, O or NH, under the proviso that not two heteroatoms are bonded to each other, and p, r and u are independently of each other an integer between 0 and 10;
R″ represents the side chain of a natural amino acid;
x is 0, 1 or2;
n is an integer between 4 and 10;
m is an integer between 0 and 10;
Y represents OR or NR₁R₂ and wherein R, R₁ and R₂ are independently of each other hydrogen or alkyl, and
R′ represents the side chain of a natural amino acid or a radical of subformula Va, 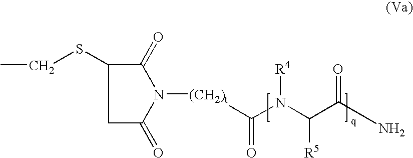
   wherein
t is an integer from 1 up to and including 10,
q is an integer from 1 up to and including 15, and
R₄ is the side chain of a natural amino acid and R₅ is hydrogen or
R₄ and R₅ together represent —(CH₂)₃—;
or a salt thereof.

Preferably, in such conjugate of formula V
A represents H, biotinyl or fluorescein-5-yl-NH—C(S)—NH—CH₂-Dᵣ-Eᵤ-Gₚ-CH₂—C(O)—, wherein D, E and G are independently of each other selected from CH₂, O or NH, under the proviso that not two heteroatoms are bonded to each other, and p, r and u are independently of each other an integer between 0 and 10;
R″ represents H or CH₂OH;
x is 0, 1 or 2;
n is 5, 6, 7 or 8;
m is 0 or 1; and
Y represents OR or NR₁R₂ and wherein R, R₁ and R₂ are independently of each other hydrogen or alkyl, and
R′ represents the side chain of a natural amino acid or a radical of subformula Va.

Preferably, in a conjugate of formula V the β-homolysine unit has the L-configuration, i.e., the structure is preferably as follows:

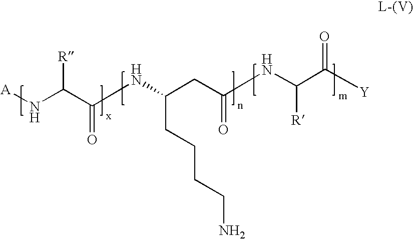

The general terms used hereinbefore and hereinafter preferably have within the context of this disclosure the following meanings, unless otherwise indicated.

The prefix “lower” denotes a radical having up to and including a maximum of 7, especially up to and including a maximum of 4 carbon atoms, the radicals in question being either linear or branched with single or multiple branching.

Where the plural form is used for conjugates, salts, and the like, this is taken to mean also a single conjugate, salt, or the like. Any asymmetric carbon atoms may be present in the (R)—, (S)— or (R,S)-configuration, preferably in the (R)— or (S)-configuraulon. The conjugates may thus be present as mixtures of isomers or as pure isomers, preferably as enantiomer-pure diastereomers. The invention relates also to possible tautomers of the conjugates described herein.

In the preferred embodiment, alkyl has up to a maximum of 12 carbon atoms and is especially lower alkyl.

Lower alkyl is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or preferably methyl.

If the CARGO is employed for non-therapeutical purposes, A is preferably biotinyl, fluorescein-5-yl-NH—C(S)— or fluorescein-5-yl-NH—C(S)—NH—CH₂-Dᵣ-Eᵤ-Gₚ-CH₂—C(O)—, wherein D, E and G are independently of each other selected from CH₂, O or NH, under the proviso that not two heteroatoms are bonded to each other, and p, r and u are independently of each other an integer between 0 and 10.

Preferably, the SHUTTLE comprises between 4 and 25, preferably between 5 and 10, β-homolysine residues, i.e. n is between 4 and 25, preferably between 5 and 10. More preferably, n is 5, 6, 7 or 8.

m is preferably an integer between 0 and 5, especially 0 or 1.

R′ is preferably —(CH₂)ₖ—SH, wherein k is an integer between 0 and 10, preferably between 0 and 4, e.g., 1.

Y is preferably NR₁R₂ and R₁ and R₂ are preferably H.

t is preferably an integer between 1 and 5, e.g. 1, 2, 3, 4 or 5.

q is preferably an integer between 1 and 12, e.g. 8.

The term “natural amino acids” as used herein means, in particular, glycine, alanine, valine, leucine, isoleucine, phenylalanine, serine, threonine, cysteine, methionine, tryptophane, tyrosine, asparagine, glutamine, asparagic acid, glutaminic acid, lysine, arginine and histidine. Preferably, the term “natural amino acids” relates to glycine, L-alanine, L-valine, L-leucine, L-isoleucine, L-phenylalanine, L-serine, L-threonine, L-cysteine, L-methionine, L-tryptophan, L-tyrosine, L-asparagine, L-glutamine, L-aspartic acid, L-glutamic acid, L-lysine, L-arginine and L-histidine.

For non-pharmaceutical purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. In one embodiment of the invention, the conjugate is employed in the form of an acetate, trifluoroacetate or trifluoromethane sulfonate.

A conjugate of formula V wherein A is fluorescein-5-yl-NH-C(S)— or fluorescein-5-yl-NH—C(S)—NH—CH₂-Dᵣ-Eᵤ-Gₚ-CH₂—C(O)—, wherein D, E and G are independently of each other selected from CH₂, O or NH, under the proviso that not two heteroatoms are bonded to each other, and p, r and u are independently of each other an integer between 0 and 10, and Y is NH₂, may be prepared by processes that, though not applied hitherto for the new conjugates of the present invention, are known per se, especially a process characterized in that a peptide of the formula VI 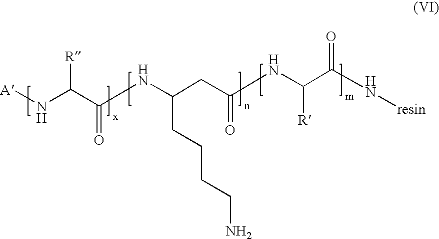
wherein A′ represents H, or H₂N—CH₂-Dᵣ-Eᵤ-Gₚ-CH₂—C(O)—, wherein D, E and G are independently of each other selected from CH₂, O or NH, under the proviso that not two heteroatoms are bonded to each other, and p, r and u are independently of each other an integer between 0 and 10; the resin is attached to the nitrogen atom with a bond that can be hydrolysed under reaction conditions that do not result in the hydrolysis of peptide bonds; and the other symbols and radicals have the meaning as defined above for a conjugate of formula V, is first reacted with isothiocyanato fluorescein in the presence of a suitable base, e.g., diisopropylethylamine in a suitable solvent, preferably N-methyl-2-pyrrolidone, at a temperature between 0° C. and 50° C., e.g., at room temperature, for a period of 6 h to 36 hours, e.g., 18, 21 or 24 hours and afterwards cleaved from the resin by treatment with a mixture of trifluoroacetic acid and water for 15 to 360 minutes, e.g., 120 minutes, at a temperature between 0° C. and 50° C., e.g., at room temperature, wherein the starting compound of formula VI may also be present with functional groups in protected form, if necessary, and/or in the form of salts, provided the reaction in salt form is possible;
wherein any protecting groups in a protected derivative of a conjugate of the formula V are removed;
and, if so desired, a free conjugate of formula V is converted into a salt, an obtainable salt of a conjugate of formula V is converted into the free conjugate or another salt, and/or a mixture of isomeric conjugates of formula V is separated into the individual isomers.

The obtained conjugate of formula V, wherein Y represents NH₂, can be further transformed into a conjugate of formula V, wherein Y represents OR or NR₁R₂, in which radicals R, R₁ and R₂ are independently of each other hydrogen or alkyl, by way of reactions known as such (see, e.g. “Solid-Phase Synthesis”, Ed. S. A. Kates and F. Albericio, 2000, Marcel Dekker, Inc., New York, Basel, or “Chemical Approaches to the Synthesis of Peptides and Proteins”, CRC Press, Boca Raton, 1997).

Protecting Groups

If one or more other functional groups, for example carboxy, hydroxy, amino, or mercapto, are or need to be protected in a conjugate of formula V, because they should not take part in the reaction, these are such groups as are usually used in the synthesis of peptide compounds, and also of cephalosporins and penicillins, as well as nucleic acid derivatives and sugars.

The protecting groups may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter.

The protection of such functional groups by such protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Wiley, New York 1981, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/1, Georg Thieme Verlag, Stuttgart 1974, in H.-D. Jakubke and H. Jescheit, “Aminosäuren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide und Derivate” (Chemistry of carbohydrates: monosaccharides and derivatives), Georg Thieme Verlag, Stuttgart 1974.

Additional Process Steps

Salts of a conjugate of formula V may be prepared in a manner known per se. Acid addition salts of conjugates of formula V may thus be obtained by treatment with an acid or with a suitable anion exchange reagent.

Salts can usually be converted to free conjugates, e.g. by treating with suitable basic agents, for example with alkali metal carbonates, alkali metal hydrogencarbonates, or alkali metal hydroxides, typically potassium carbonate or sodium hydroxide.

General Process Conditions

All process steps described here can be carried out under known reaction conditions, preferably under those specifically mentioned, in the absence of or usually in the presence of solvents or diluents, preferably such as are inert to the reagents used and able to dissolve these, in the absence or presence of catalysts, condensing agents or neutralisiing agents, for example ion exchangers, typically cation exchangers, for example in the H⁺ form, depending on the type of reaction and/or reactants at reduced, normal, or elevated temperature, for example in the range from −100° C. to about 190° C., preferably from about −80° C. to about 150° C., for example at −80 to −60° C., at room temperature, at −20 to 40° C. or at the boiling point of the solvent used, under atmospheric pressure or in a closed vessel, where appropriate under pressure, and/or in an inert atmosphere, for example under argon or nitrogen.

Salts may be present In all starting compounds and transients, if these contain salt-forming groups. Salts may also be present during the reaction of such compounds, provided the reaction is not thereby disturbed.

The solvents from which those can be selected which are suitable for the reaction in question include for example water, esters, typically lower alkyl-lower alkanoates, e.g diethyl acetate, ethers, typically aliphatic ethers, e.g. diethylether, or cyclic ethers, e.g. tetrahydrofuran, liquid aromatic hydrocarbons, typically benzene or toluene, alcohols, typically methanol, ethanol or 1- or 2-propanol, nitrites, typically acetonitrile, halogenated hydrocarbons, typically dichloromethane, acid amides, typically dimethylformamide, bases, typically heterocyclic nitrogen bases, e.g. pyridine, carboxylic acids, typically lower alkanecarboxylic acids, e.g. acetic acid, carboxylic acid anhydrides, typically lower alkane acid anhydrides, e.g. acetic anhydride, cyclic, linear, or branched hydrocarbons, typically cyclohexane, hexane, or isopentane, or mixtures of these solvents, e.g. aqueous solutions, unless otherwise stated in the description of the process. Such solvent mixtures may also be used in processing, for example through chromatography or distribution.

In the preferred embodiment, a conjugate of formula V is prepared according to or in analogy to the processes and process steps defined in the Examples.

Starting Materials

New starting materials and/or intermediates, as well as processes for the preparation thereof, are likewise the subject of this invention. In the preferred embodiment, such starting materials are used and reaction conditions so selected as to enable the preferred compounds to be obtained.

Starting materials of the formula VI are known, can be synthesized in analogy to or according to methods that are known in the art.

For example, a compound of the formula VI wherein m is 0 can be prepared by first coupling the amino acid β-homolysine in protected form (VII), 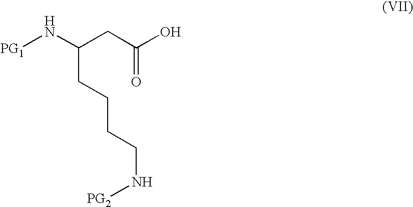
wherein PG₁ is a protection group, preferably fluoren-9-yl-methoxycarbonyl, and PG₂ is a protection group, preferably tert-butoxycarbonyl, to a resin (VIII)
H₂N-resin (VIII)
having a side chain comprising at least one secondary amine covalently attached to such side chain by a bond that can be hydrolysed under reaction conditions that do not result in the hydrolysis of peptide bonds, in the presence of a suitable base, e.g., diisopropylethylamine in a suitable solvent, preferably N-methyl-2-pyrrolidone, at a temperature between 0° C. and 50° C., e.g., at room temperature, for a period of 60 to 180 minutes, e.g., 90 minutes, in the presence of between 1 and 5 equivalents of an coupling agent, e.g., O-(1,2-dihydro-2-oxo-1-pyridyl)-1,1,3,3-tetramethyluronium tetrafluoroborate, N-[(dimethylamino)1H-1,2,3-triazolo[4,5-b]pyridin-1-ylmethylene]-N-methylmethanaminium hexafluorophosphate N-oxide or tetramethylfluoroformamidinium hexafluorophosphate, in order to provide the coupling product (IX), 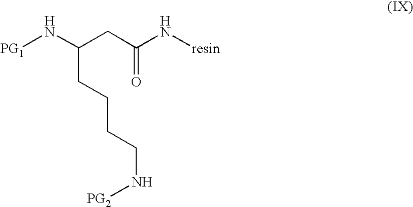
wherein PG₁ is a protection group, preferably fluoren-9-yl-methoxycarbonyl and PG₂ is a protection group, preferably tert-butoxycarbonyl.

In a second step, the protection group PG₁ is detached under suitable reaction conditions from the coupling product (IX) providing a compound of formula (X), 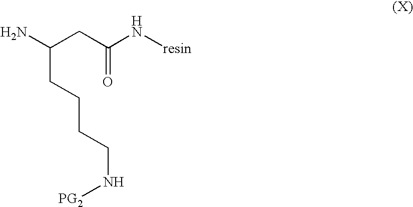
wherein PG₂ is a protection group, preferably tert-butoxycarbonyl.

To such compound of formula (X) further β-homolysine units are added step-by-step by repeating the reaction sequence of first adding a further β-homolysine unit in protected form (VII) as described above under reaction conditions identical or similar to those described for the coupling reaction between the β-homolysine unit (VII) and the resin (VIII) and secondly detaching the protection group PG₁ is under suitable reaction conditions from the coupling product, providing finally a compound of formula (XI), 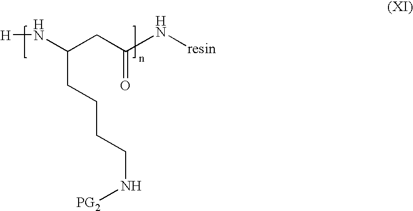
wherein PG₂ is a protection group, preferably tert-butoxycarbonyl.

If in a compound of formula (VI) x is different from 0, to such compound of formula (XI) further protected amino acids of formula XII 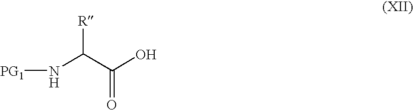
wherein PG₁ is a protecting group, preferably fluoren-9-yl-methoxycarbonyl, and R″ has the meaning as provided for a compound of formula V, respectively, are added step-by-step by repeating the reaction sequence of first adding an protected amino acid of formula (XII) under reaction conditions identical or similar to those described for the coupling reaction between the β-homolysine unit (VII) and the resin (VIII) and secondly detaching the protection group PG₁ is under suitable reaction conditions from the coupling product, providing finally a compound of formula (XIII), 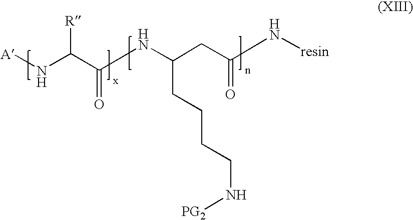
wherein A′ is hydrogen, PG₂ is a protection group, preferably tert-butoxycarbonyl, and x and n have the meanings as provided above for a compound of formula V.

If in a compound of formula VI, A′ shall represent H₂N—CH₂-Dᵣ-Eᵤ-GᵣCH₂—C(O)—, wherein D, E and G are independently of each other selected from CH₂, O or NH, under the proviso that not two heteroatoms are bonded to each other, and p, r and u are independently of each other an integer between 0 and 10, the compound of formula (XIII) is further reacted with a PG₁-protected acid of formula (XIV),
PG₁-HN—CH₂-Dᵣ-Eᵤ-Gₚ-CH₂—C(O)—OH (XIV)
wherein PG₁ is a protecting group, preferably fluoren-9-yl-methoxycarbonyl, and D, E and G are independently of each other CH₂, O or NH, under the proviso that not two heteroatoms are bonded to each other, and p, r and u are independently of each other an integer between 0 and 10, by the reaction sequence of first adding an PG₁-protected acid of formula (XIV) under reaction conditions identical or similar to those described for the coupling reaction between the β-homolysine unit (VII) and the resin (VIII) and secondly detaching the protection group PG₁ under suitable reaction conditions from the coupling product, providing a compound of formula (VI) wherein m is 0, 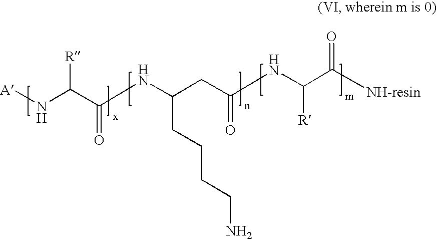
wherein A′ represents H, or H₂N—CH₂-Dᵣ-Eᵤ-Gₚ-CH₂—C(O)—, wherein D, E and G are independently of each other selected from CH₂, O or NH, under the proviso that not two heteroatoms are bonded to each other, and p, r and u are independently of each other an integer between 0 and 10; and the other symbols and radicals have the meaning as defined above for a conjugate of formula V.

If in a compound of formula (VI) m shall be different to 0, protected amino acids of formula XII wherein PG₁ is a protecting group, preferably fluoren-9-yl-methoxycarbonyl, and R′ has the meaning as provided for a compound of formula V, respectively, are added to the resin of formula (VIII), step-by-step by repeating the reaction sequence of first adding an protected amino acid of formula (XII) under reaction conditions identical or similar to those described for the coupling reaction between the β-homolysine unit (VII) and the resin (VIII) and secondly detaching the protection group PG₁ is under suitable reaction conditions from the coupling product, providing the coupling product (XVI) 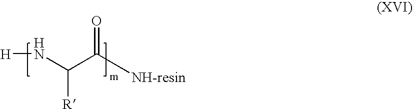
wherein R′ and m have the meanings as provided above for a compound of formula V, and employing instead of the resin of formula (VIII) such coupling product (XVI) as a starting material for the reaction sequence described above.

A conjugate of formula V wherein A represents an oligonucleotide, peptide, protein, a diagnostic imaging or contrast agent, or biotinyl can be obtained by starting from a compound of formula VI wherein A′ represents H and the other symbols and radicals have the meaning as defined above for a conjugate of formula V and applying reactions known as such in the art (see, e.g., P. Lloyd-Williams, F. Albericio and E. Giralt in “Chemical Approaches to the Synthesis of Peptides and Proteins, CRC Press, Boca Raton, 1997, e.g. in Ch. 4.4, pp. 175 -207, and in the publications cited therein). In particular, a conjugate of formula V wherein A represents biotinyl can be prepared by reacting a compound of formula VI wherein A′ represents H and the other symbols and radicals have the meaning as defined above for a conjugate of formula V, with biotin (XVI), 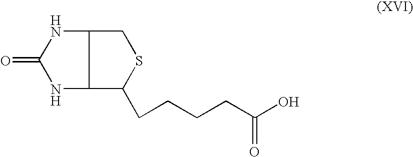
under reaction conditions identical or similar to those described above for the coupling reaction between the β-homolysine unit (VII) and the resin (VII).

The methods described before provide a compound of formula V wherein R′ represents the side chain of a natural amino acid. In order to obtain a compound of formula V wherein R′ represents a radical of subformula Va, 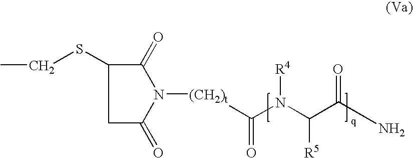
wherein t is an integer from 1 up to and including 10, q is an integer from 1 up to and including 15, and R₄ is the side chain of a natural amino acid and R₅ is hydrogen or R₄ and R₅ together represent —(CH₂)₃—, one of the rests R′ introduced by the methods described above has to represent —CH₂—SH, i.e. an amino acid cysteine has to be added to the peptide coupled to the resin at least once. To such compound of formula V comprising at least one rests R′ representing —CH₂—SH, the method described by R. Eritja on page 535 to page 537 of “Solid-Phase Synthesis”, Ed. S. A. Kates and F. Albericio, 2000, Marcel Dekker, Inc., New York, Basel, Ch. 12, or similar methods can be applied.

In the preparation of starting materials, existing functional groups which do not participate in the reaction should, if necessary, be protected. Preferred protecting groups, their introduction and their removal are described under “protecting goups” or in the Examples.

All remaining starting materials of are known, capable of being prepared according to known processes, or commercially obtainable; in particular, they can be prepared using processes as described in the Examples.

In therapeutical applications, the dosage of the conjugates depends upon a variety of factors including the CARGO employed, type, species, age, weight, sex and medical condition of the patient; the severity of the condition to be treated; the route of administration and the renal and hepatic function of the patient. A physician, clinician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug.

The invention relates also to pharmaceutical compositions comprising an effective amount, especially an amount effective in the treatment of one of the below-mentioned diseases, of a CONJUGATE together with pharmaceutically acceptable carriers that are suitable for topical, enteral, for example oral or rectal, or parenteral administration and that may be inorganic or organic, solid or liquid. There are used for oral administration especially tablets or gelatin capsules that comprise the active ingredient together with diluents, for example lactose, dextrose, mannitol, and/or glycerol, and/or lubricants and/or polyethylene glycol. Tablets may also comprise binders, for example magnesium aluminum silicate, starches, such as corn, wheat or rice starch, gelatin, methylcellulose, sodium carboxymethylcellulose and/or polyvinylpyrrolidone, and, if desired, disintegrators, for example starches, agar, alginic acid or a salt thereof, such as sodium alginate, and/or effervescent mixtures, or adsorbents, dyes, flavorings and sweeteners. It is also possible to use the pharmacologically active conjugates of the present invention in the form of parenterally administrable compositions or in the form of infusion solutions. The pharmaceutical compositions may be sterilized and/or may comprise excipients, for example preservatives, stabilisers, wetting agents and/or emulsifiers, solubilisers, salts for regulating the osmotic pressure and/or buffers. The present pharmaceutical compositions, which may, if desired, comprise other pharmacologically active substances are prepared in a manner known per se, for example by means of conventional mixing, granulating, confectioning, dissolving or lyophilising processes, and comprise approximately from 1% to 95%, especially from approximately 1% to approximately 20%, active ingredient(s).

Furthermore, the invention relates to a pharmaceutical composition for treatment of tumours in warm-blooded animals, including humans, comprising an antitumourally effective dose of a conjugate comprising a cytotoxic CARGO or a pharmaceutically acceptable salt of such a conjugate together with a pharmaceutical carrier.

Another aspect of the present invention relates to the use of a CONJUGATE or a pharmaceutically acceptable salt thereof in a method for the treatment of the human or animal body and in the manufacture of a medicament for the treatment of an infectious disease, e.g. an HIV-infection, epilepsy, anxiety, pain, psychosis, schizophrenia, migraine, depression, Alzheimer's disease, Parkinson's disease, arthritis (e.g. osteoarthritis and rheumatoid arthritis), tissue ulceration (e.g. comeal, foot ulcerations, epidermal and gastric ulceration), abnormal wound healing, periodontal disease, bone disease (e.g. Paget's disease and osteoporosis), psoriasis, artherosclerosis, diabetes, hyperglycemia, hyperinsulinaemia, hyperlipidaemia, insulin resistance, impaired glucose metabolism, obesity, diabetic retinopathy, macular degeneration, cataracts, diabetic nephropathy, glomerulosclerosis, diabetic neuropathy, erectile dysfunction, premenstrual syndrome, polycystic ovarian syndrome, vascular restenosis, coronary heart disease, hypertension, angina pectoris, myocardial infarction, stroke, skin and connective tissue disorders, metabolic acidosis, conditions of impaired glucose tolerance, allograft transplant rejection, allergic diseases, asthma and, in particular, proliferative diseases, like liquid (e.g., leukemia) and solid tumor diseases.

The term usolid tumor disease” especially means ovarian cancer, breast cancer, thyroid cancer, cancer of the colon and generally the GI tract, cervix cancer, lung cancer, e.g. small-cell lung cancer and non-small-cell lung cancer, head and neck cancer, bladder cancer, cancer of the prostate, melanoma, or Kaposi's sarcoma and relates, in particular, also to tumor metastasis.

Additionally, the present invention provides a method for delivery of a CARGO into or across a biological barrier, e.g., the skin or the blood brain barrier, the method comprising contacting the barrier with a CONJUGATE.

The following Examples serve to illustrate the invention without limiting the invention in its scope.

Temperatures are measured in degrees celsius (° C.). Unless otherwise indicated, the reactions take place at room temperature.

Abbreviations

Adoa 8-amino-3,6-dioxaoctanoic acid
Ala alanine
Arg arginine
BOC tert-butoxycarbonyl
calc. calculated
Cys cysteine
DMSO dimethylsulfoxide
FITC fluorescein isothiocyanate
Fmoc fluoren-9-yl-methoxycarbonyl
Gly glycine
HPLC high pressure liquid chromatography
Leu leucine
LSC laser scanning cytometer
Lys lysine
Me methyl
Phe phenylalanine
PBS/O phosphate buffered saline without calcium
Pro proline
Ser serine
Ser(tBu) O-tert-butyl-serine
TFA trifluoroacetic acid
t_{R} retention time
   Analytical HPLC

Gradient 1, linear gradient over 7 min of MeCN/0.09% TFA and H₂O/0.1% TFA from 1:49 to 1:0 and 3 min at 1:0; flow rate 2.0 mL/min, detection at 215 nm; SMT C₁₈ column (250×4.6 mm; 5 μm, 100 Å). Gradient 2, linear gradient over 10 min of MeCN/0.09% TFA and H₂O/0.1% TFA from 1:49 to 3:2; flow rate 2.0 mL/min, detection at 215 nm; SMT C₁₈ column (250×4.6 mm; 5 μm, 100 Å). Gradient 3, linear gradient from over 2.5 min of MeCN/0.09% TFA and H₂O/0.1% TFA from 1:49 to 3:2; flow rate 4 ml/min; detection at 215 nm; Chromolith Speed ROD C₁₈ column (50×4.6 mm).

### EXAMPLE 1

### N-(Fluorescein-5-yl)-thioureido-N′-Adoa-(β-homolysine)₇-NH₂ TFA salt

The target β-peptide is synthesised manually on a 4-(2′,4′-dimethoxyl-phenyl-aminomethyl)-phenoxy resin (f=0.53 mmol/g; NovaBiochem, Läufelfingen, Switzerland), employing the Fmoc strategy and protocols known in the art (see, E. Atherton and R. C. Sheppard, In Rickwoood, D. and Hames, B. D. (Eds) Solid phase peptide synthesis, a practical approach, Oxford University Press, Oxford, 1990).

The complete β-peptide resin from step 1.6 is deprotected and cleaved by treatment with trifluoroacetic adid/water (95:5, v/v) for 2 h at room temperature. The filtrate from the cleavage reaction is precipitated in diisopropyl ether—petroleum ehter (1:1, v/v, 0° C.), and the precipitate is collected by filtration. The crude compound is purified by reversed-phase medium-pressure liquid chromatography using a C₁₈ column eluted with an acetonitrile-water gradient containing 0.1% trifluoroacetic acid (Merck, LICH ROPREP RP-18, 15-25 μm bead diameter, reversed phase column material based on C₁₈-derivatised silicagel, Merck, Darmstadt, FRG; column length 46 cm, diameter 3.6 cm; flow rate 53.3 m/min; detection at 215 nm). Mass spectrometric analysis (matrix-assisted laser-desorption ionization time-of-flight mass spectrometry, MALDI-TOF) of the purified compound reveals molecular masses within 0.1% of the expected value (negative ion mode): 1546.9 (calc. 1546.0). The purity of the title compound is verified by reversed-phase analytical HPLC: single peak at t_{R}=5.13 min. (Gradient 1).

Step 1.1:

N^{β}-Fmoc-N^{ω}-Boc-L-β-homolysine (2 equiv.; Fluka, Buchs, Switzerland) is coupled with O-(1,2-dihydro-2-oxo-1-pyridyl)-1,1,3,3-tetramethyluronium tetrafluoroborate (2.0 equiv.) in the presence of diisopropylethylamine (2.2 equiv.). Coupling is achieved by first dissolving the Fmoc-β-homolysine derivative, the base, and the coupling agent in N-methyl-2-pyrrolidone, then waiting 3 min for preactivation, adding the mixture to the resin, and finally shaking at room temperature for at least 90 min. If required, a second coupling is performed by using N-[(dimethylamino)1H-1,2,3-triazolo[4,5-b]pyridin-1-ylmethylene]-N-methylmethanaminium hexafluorophosphate N-oxide (2.0 equiv.) or tetramethylfluoroformamidinium hexafluorophosphate (2.0 equiv) as coupling agent in the presence of diisopropylethylamine (6 equiv.).

Step 1.2:

After the amino coupling step, a capping procedure is performed with acetic anhydride:pyridine:dimethylacetamide (1:1:1, v/v/v) to prevent the formation of deletion sequences.

Step 1.3:

The Fmoc protection group is removed from the product of step 1.2 with piperidine/dimethylacetamide (1:4, v/v; 8×2 min), followed by washing with isopropanol (3×1 min), N-methyl-2-pyrrolidone (3×2 min), isopropanol (3×1 min), and N-methyl-2-pyrrolidone (3×2 min).

Step 1.4:

Steps 1.1 to 1.3 are repeated for 6 times providing a peptide attached to the resin mentioned above comprising seven β-homolysine units.

Step 1.5:

Fmoc-8-amino-3,6-dioxaoctanoic acid (Neosystem, Strasbourg, France) is coupled as described for the Fmoc-β-lysine derivative under step 1.1 and the Fmoc protection group is removed as described under step 1.3.

Step 1.6:

5-Isothiocyanato fluorescein (FITC, “Isomer I”, 3 equiv.; Fluka, Buchs, Switzerland) is incorporated to the N-terminal amino group in the presence of diisopropylethylamine (6 equiv.). Coupling is achieved by dissolving the building block and the base in N-methyl-2-pyrrolidone, adding the mixture to the resin, and shaking at room temperature for 21 h.

### EXAMPLE 2

### N-(Fluorescein-5-yl)-thioureido-N′-Adoa-(β-homolysine)₅-NH₂ TFA salt

The title compound is obtained analog to Example 1. Title compound: mass spectral analysis (negative ion mode)=1261.7 (calc. 1261.6), t_{R}=4.96 min (Gradient 1).

### EXAMPLE 3

### N-(Fluorescein-5-yl)-thioureido-N′-Adoa-(β-homolysine)₈-NH₂ TFA salt

The title compound is obtained analog to Example 1. Title compound: mass spectral analysis (negative ion mode)=1403.9 (calc. 1403.8), t_{R}=5.16 min (Gradient 1).

### EXAMPLE 4

### N-(Fluorescein-5-yl)-thioureido-N′-Adoa-(β-homolysine)₈-NH₂ TFA salt

The title compound is obtained analog to Example 1. Title compound: mass spectral analyis (negative ion mode)=1687.3 (calc. 1688.2), t_{R}=5.13 min (Gradient 1).

### EXAMPLE 5

### Biotin-Ser-Gly-(β-homolysine)₆-NH₂ TFA salt

The title compound is obtained analog to Example 1. N^{α}-Fmoc-Gly-OH, N^{α}-Fmoc-Ser(tBu)-OH and (+)-biotin (Fluka, Buchs, Switzerland) are coupled as described in Example 1. Title compound: mass spectral analyis (positive ion mode)=1241.8 (calc. 1241.7), t_{R}=5.54 min (Gradient 2).

### EXAMPLE 6

### Biotin-Ser-Gly-(β-homolysine)₇-NH₂ TFA salt

The title compound is obtained analog to Example 1. N^{α}-Fmoc-Gly-OH, N^{α}-Fmoc-Ser(tBu)-OH and (+)-biotin (Fluka, Buchs, Switzerland) are coupled as described in Example 1. Title compound: mass spectral analyis (positive ion mode)=1383.4 (calc. 1383.9), t_{R}=5.62 min (Gradient 2).

### EXAMPLE 7

### Biotin-Ser-Gly-(β-homolysine)₈-NH₂ TFA salt

The title compound is obtained analog to Example 1. N^{a}-Fmoc-Gly-OH, N^{a}-Fmoc-Ser(tBu)-OH and (+)-biotin (Fluka, Buchs, Switzerland) are coupled as described in Example 1. Title compound: mass spectral analysis (positive ion mode)=1526.3 (calc. 1526.1), t_{R}=5.65 min (Gradient 2).

### EXAMPLE 8

### N-(Fluorescein-5-yl)-thioureido-N′-Adoa-(β-homolysine)B-Cys-NH₂ TFA salt

The title peptide is synthesised on a Milligen 9050 automated peptide synthesiser (continuos flow; Millipore, Bedford, Mass. USA) in analogy to the method described under Example 1, starting with an Fmoc-PAL-PEG-PS resin (see F. Albericio et al., J.Org.Chem., 55 (1990) 3730-3743) for establishing the C-terminal carboxamide, and using protocols based on the fluorenylmethoxycarbonyl chemistry (see, E. Atherton and R. C. Sheppard, in Rickwoood, D. and Hames, B. D. (Eds) Solid phase peptide synthesis, a practical approach, Oxford University Press, Oxford, 1990). N^{α}-Fmoc-Cys(Trt) (3 equiv.) is incorportated using its 2,4,5-trichlorophenyl ester (single coupling) with minimum reaction time of 30 min (see 9050 Plus PepSynthesizer User's Guide, Millipore Corporation, Bedford, Mass. 1992). The required N^{β}-Fmoc-N^{α}-Boc-L-b-homolysine (3 equiv.; Fluka, Buchs, Switzerland) Is coupled with O(1,2-dihydro-2-oxo-1-pyridyl)-1,1,3,3-tetramethyluronium tetrafluoroborate (3.0 equiv.) in the presence of diisopropylethylamine (6.0 equiv.). The N-terminal fluorescein group is incorporated as described in Example 1. The complete β-peptide resin is deprotected and cleaved by treatment with trifluoroacetic acid/water (95:5, v/v) for 3 h at room temperature. The filtrate from the cleavage reaction is precipitated in diisopropyl ether—petroleum ether (1:1, v/v, 0° C.), and the precipitate is collected by filtration. The crude compound is purified by reversed-phase medium-pressure liquid chromatography as described in Example 1. Title compound: mass spectral analysis (negative ion mode)=1506.7 (calc. 1506.9), t_{R}=1.75 min (Gradient 3).

### EXAMPLE 9

### N-(Fluorescein-5-yl)-thioureido-N′-Adoa-(β-homolysine)₇-Cys-NH₂ TFA salt

The title compound is prepared as described in Example 8. Title compound: mass spectral analysis (negative ion mode)=1648.1 (calc. 1649.1), t_{R}=1.74 min (Gradient 3).

### EXAMPLE 10

### N-(Fluorescein-5-yl)-thioureido-N′-Adoa-(β-homolysine)₈-Cys-NH₂ TFA salt

The title compound is prepared as described in Example 8. Title compound: mass spectral analysis (negative ion mode)=1791.1 (calc. 1791.3), t_{R}=1.72 min (Gradient 3).

### EXAMPLE 11

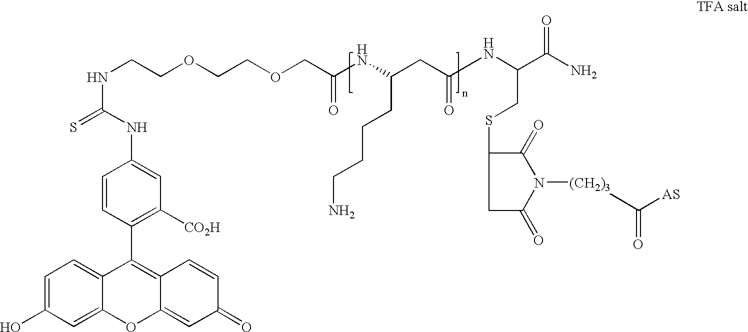 wherein AS means Pro-Ala-Lys-Arg-Lys-Leu-Phe-Gly-NH₂ and n is 6.

70.0 mg of N-(Fluorescein-5-yl)-thioureido-N′-Adoa-(β-homolysine)₆-Cys-NH₂ (Example 8) and 43.3 mg of 4-maleimido-butyryl-Pro-Ala-Lys-Arg-Lys-Leu-Phe-Gly-NH₂ (step 11.1) are dissolved in 0.46 ml of water/acetonitrile (3:1 v/v). The reaction mixture is stirred for 2 h at room temperature and 41 h at 45° C. and purified by reversed-phase liquid chromatography. Title compound: mass spectral analysis (positive ion mode)=2589.3 (calc. 2589.3), t_{R}=1.88 min (Gradient 3).

Step 11.1: 4-Maleimido-butyryl-Pro-Ala-Lys-Arg-Lys-Leu-Phe-Gly-NH₂

The title compound is synthesised on a Milligen 9050 automated peptide synthesiser (continues flow; Millipore, Bedford, Mass. USA) as described in Example 8. The required Fmoc amino acids (3 equiv.) are coupled using their 2,4,5-trichlorophenyl esters with minimum reaction times of 30 min. Side chains are protected with the following groups: tertbutoxycarbonyl for lysine and 2,2,5,7,8-pentamethyl-chroman-6-sulfonyl for arginine. 4-Maleimidobutyric acid (3 equiv.; Buchs, Switzerland) is coupled with O-(1,2-dihydro-2-oxo-1-pyridyl)-1,1,3,3-tetramethyluronium tetrafluoroborate (3.0 equiv.) in the presence of diisopropylethylamine (6.0 equiv.). The cleavage from the solid support and purification of the title compound are performed as described in Example 8. Title compound: mass spectral analysis (negative ion mode)=1079.3 (calc. 1079.3), t_{R}=1.46 min (Gradient 3).

### EXAMPLE 12

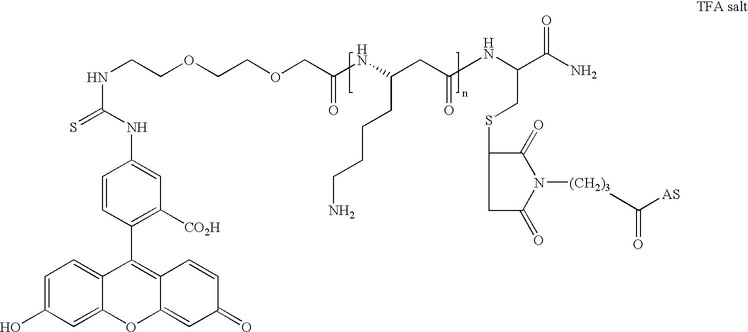 wherein AS means Pro-Ala-Lys-Arg-Lys-Leu-Phe-Gly-NH₂ and n is 7.

The title compound is obtained analog to Example 11 using N-(Fluorescein-5-yl)-thioureido-N′-Adoa-(β-homolysine)₇-Cys-NH₂ (Example 9). Title compound: mass spectral analysis (positive ion mode)=2731.0 (calc. 2731.5), t_{R}=1.87 min (Gradient 3).

### EXAMPLE 13.

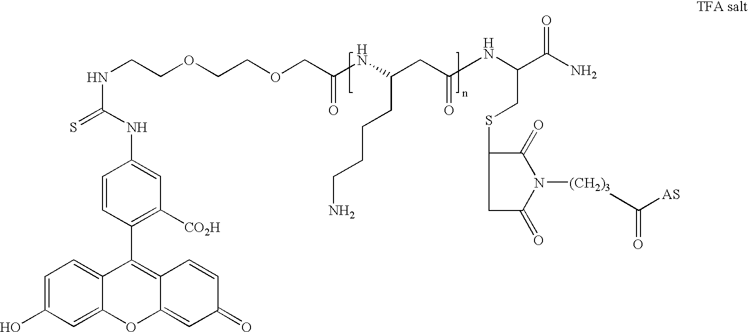 wherein AS means Prn-Ala-Lys-Arg-Lys-Leu-Phe-Gly-NH₂ and n is 8.

The title compound is obtained analog to Example 11 using N-(Fluorescein-5-yl)-thioureido-N′-Adoa-(β-homolysine)₈-Cys-NH₂ (Example 10). Title compound: mass spectral analysis (positive ion mode)=2872.7 (calc. 2873.7), t_{R}=1.86 min (Gradient 3).

### EXAMPLE 14

### Assessment of Intracellular Delivery and Nuclear Accumulation of the CONJUGATES in DU145 and HCT15 cells

CONJUGATES are diluted to 10 mM stock solutions in PBS/O. FITC is dissolved in DMSO. Exponentially growing DU145 and HCT15 cells are treated for 18 h with increasing concentrations (0.1, 1 and 10 μM) of the FITC-labelled CONJUGATES of Examples 1, 2, 3 and 4. As controls, additional aliquots of cells are incubated with the same concentrations of fluorescein. Cells are harvested, fixed and mounted on microscope slides following standard procedures. After treatment with 100 μg/ml RNAs A, slides are coverslipped with a 50% glycerol/PBS solution containing 0.2 pg/ml propidium iodide (PI).

The fluorescence emission of the stained cells is measured using the LSC. The slides are scanned using a 20× objective and an argon-ion laser operating at 5 mW and at the 488-nm line. A minimum of 5000 cells are examined. The contouring parameter is the long red fluorescent signal of PI and a 100 minimum pixel area threshold is used. Red and green fluorescence are collected by separate photomultipliers. To estimate the relative nuclear accumulation of FITC-labeled compounds, the background gate is defined using the values of the green fluorescence intensity within the countouring area of the control cells treated with fluorescein alone.

The obtained results document clearly that contrary to the control fluorescein all tested CONJUGATES are accumulating in the cells and especially in the cell nucleus proportionally to their concentration employed in the test.

1. A conjugate that comprises a) at least one compound (CARGO) to be delivered into or across a biological barrier; b) a delivery-enhancing transporter (SHUTTLE) comprising a β-homolysine polymer comprising at least 4 β-homolysine residues; c) optionally a linker (LINKER) between the components a) and b); and d) optionally a labelling unit (A); or a salt thereof.

2. A conjugate according to claim 1 having a structure selected from the group of structures (I) to (V),
A-SHUTTLE-CARGO-(CO)—Y (I),
A-CARGO-SHUTTLE-(CO)—Y (II),
SHUTTLE-LINKER-CARGO (III), and
SHUTTLE-LINKER-CARGO-(CO)—Y (IV),
wherein Y is OR or NR₁R₂ and wherein R, R₁ and R₂ independently of each other represent hydrogen or alkyl; or a salt thereof.

3. A conjugate according to claim 1 that comprises a delivery-enhancing transporter comprising between 4 and 25 β-homolysine residues; or a salt thereof.

4. A conjugate according to claim 1 that comprises a delivery-enhancing transporter comprising between 5 and 10 β-homolysine residues; or a salt thereof.

5. A conjugate according to claim 1 wherein A is selected from biotinyl, fluorescein-5-yl and fluorescein-5-yl-NH—C(S)—NH—CH₂-Dᵣ-Eᵤ-Gₚ-CH₂—C(O)—, wherein D, E and G are independently of each other selected from CH₂, O or NH, under the proviso that not two heteroatoms are bonded to each other, and p, r and u are independently of each other an integer between 0 and 10; or a salt thereof.

6. A conjugate according to claim 1 wherein the CARGO is a biomolecule selected from the group consisting of oligonucleotides, peptides and proteins; or a salt thereof.

7. conjugate according to claim 1 wherein the CARGO is an antibody; or a salt thereof.

8. A conjugate according to claim 1 wherein the CARGO is pharmacologically active compound; or a salt thereof.

9. A conjugate according to claim 1 wherein the CARGO is a diagnostic imaging or contrast agent; or a salt thereof.

10. A conjugate according to claim 1 represented by formula V 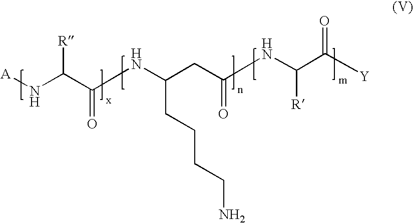
wherein
A represents an oligonucleotide, peptide, protein, a diagnostic imaging or contrast agent, H, biotinyl, fluorescein-5-yl-NH—C(S)— or fluorescein-5-yl-NH—C(S)—NH—CH₂-Dᵣ-Eᵤ-Gₚ-CH₂—C(O)—, wherein D, E and G are independently of each other selected from CH₂, O or NH, under the proviso that not two heteroatoms are bonded to each other, and p, r and u are independently of each other an integer between 0 and 10;
R″ represents the side chain of a natural amino acid;
x is 0, 1 or 2;
n is an integer between 4 and 10;
m is an integer between 0 and 10;
Y represents OR or NR₁R₂ and wherein R, R₁ and R₂ are independently of each other hydrogen or alkyl, and
R′ represents the side chain of a natural amino acid or a radical of subformula Va, 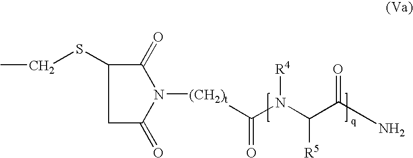
wherein
t is an integer from 1 up to and including 10,
q is an integer from 1 up to and including 15, and
R₄ is the side chain of a natural amino acid and R₅ is hydrogen or
R₄ and R₅ together represent —(CH₂)₃—;
or a salt thereof.

11. A conjugate according to claim 10 of formula V wherein
A represents H, biotinyl or fluorescein-5-yl-NH—C(S)—NH—CH₂-Dᵣ-Eᵤ-Gₚ-CH₂—C(O)—, wherein D, E and G are independently of each other selected from CH₂, O or NH, under the proviso that not two heteroatoms are bonded to each other, and p, r and u are independently of each other an integer between 0 and 10;
R″ represents H or CH₂OH;
x is 0, 1 or2;
n is 5, 6, 7 or 8;
m is 0 or 1; and
Y represents OR or NR₁R₂ and wherein R, R₁ and R₂ are independently of each other hydrogen or alkyl, and
R′ represents the side chain of a natural amino acid or a radical of subformula Va, wherein
p is an integer from 1 up to and including 10,
q is an integer from 1 up to and including 15, and
R₄ is the side chain of a natural amino acid and R₅ is hydrogen or
R₄ and R₅ together represent —(CH₂)₃—;
or a salt thereof.

12. A pharmaceutical composition comprising a conjugate according to claim 1 together with at least one pharmaceutically acceptable carrier.

13. A conjugate according to claim 1 or a pharmaceutically acceptable

14. A method for delivery of a compound (CARGO) into or across a biological barrier, the method comprising contacting the barrier with a conjugate according to claim 1.

15. The method according to claim 14 wherein the biological barrier is skin or the blood brain barrier.

16. A process for the preparation of a conjugate of formula V wherein A is fluorescein-5-yl-NH—C(S)— or fluorescein-5-yl-NH—C(S)—NH—CH₂-Dᵣ-Eᵤ-Gₚ-CH₂—C(O)—, wherein D, E and G are independently of each other selected from CH₂, O or NH, under the proviso that not two heteroatoms are bonded to each other, and p, r and u are independently of each other an integer between 0 and 10, Y is NH₂, and the other symbols and radicals have the meaning as defined in claim 10 for a conjugate of formula V, wherein a peptide of the formula VI 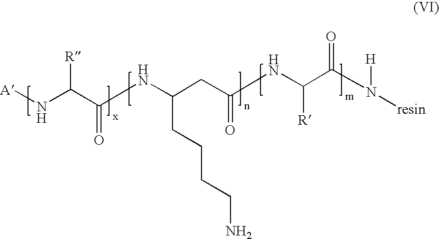
wherein A′ represents H, or H₂N—CH₂-Dᵣ-Eᵤ-Gₚ-CH₂—C(O)—, wherein D, E and G are independently of each other selected from CH₂, O or NH, under the proviso that not two heteroatoms are bonded to each other, and p, r and u are independently of each other an integer between 0 and 10; the resin is attached to the nitrogen atom with a bond that can be hydrolysed under reaction conditions that do not result in the hydrolysis of peptide bonds; and the other symbols and radicals have the meaning as defined in claim 10 for a conjugate of formula V,
is first reacted with isothiocyanato fluorescein and afterwards cleaved from the resin,
wherein the starting compound of formula VI may also be present with functional groups in protected form, if necessary, and/or in the form of salts, provided the reaction in salt form is possible;
wherein any protecting groups in a protected derivative of a conjugate of the formula V are removed;
and, if so desired, a free conjugate of formula V is converted into a salt, an obtainable salt of a conjugate of formula V is converted into the free conjugate or another salt, and/or a mixture of isomeric conjugates of formula V is separated into the individual isomers.
